(19) United States
(12) Patent Application Publication
(10) Pub. No.: US 2015/0057538 A1
(43) Pub. Date: Feb. 26, 2015

(54) METHOD AND APPARATUS FOR MONITORING TOTAL DELIVERED DOSE OF CONTRAST MEDIA

(71) Applicant: Intersect Partners, LLC, Incline Village, NV (US)

(72) Inventors: Andrew H. Cragg, Edina, MN (US); Gerard Von Hoffmann, Coto de Caza, CA (US)

(21) Appl. No.: 14/466,648

(22) Filed: Aug. 22, 2014

Related U.S. Application Data
(60) Provisional application No. 61/869,006, filed on Aug. 22, 2013.

(51) Int. Cl. A61M 5/00

(52) U.S. Cl. CPC A61M 5/007
USPC 600/431

(57) ABSTRACT

Disclosed are methods and devices for monitoring total delivered dose of an analyte of interest, such as an iodine containing contrast media used during intravascular procedures. The device includes a flow rate measuring capability, and an analyte concentration measuring capability. Total cumulative delivered dose of the analyte can be determined, without regard to the number of sources of analyte and variations in concentration of the analyte delivered over the course of an intravascular procedure.

### CROSS REFERENCE TO RELATED APPLICATIONS

This application claims the priority benefit under 35 U.S.C. §119(e) of U.S. Provisional Patent Application No. 61/869,006, filed Aug. 22, 2013, the entirety of which is hereby incorporated by reference herein.

The present invention relates to fluid flow monitoring, and, in particular, to the real time determination of the total cumulative delivered dose of contrast agent such as to facilitate fluoroscopic imaging.

### BACKGROUND OF THE INVENTION

Radiopaque contrast agents increase the absorption of X-rays and result in a positive contrast in X-ray fluoroscopy (i.e., an opaque image or shadow where the radiopaque contrast agent is present). Radiopaque contrast agents typically may include, any soluble or insoluble compound that increases absorption of X-rays. Some variations of radiopaque contrast agents may comprise iodine. For example, a contrast agent may comprise an aqueous solution of one or more iodine-containing salts, such as: a salt of a diatrizoate (e.g., sodium diatrizoate such as HYPAQUE™ contrast medium, or sodium meglumine diatrizoate such as RENOGRAFIN-76™ contrast medium); a salt of 5-acetamido-2,4,6-triiodo-N-methylisophthalamic acid (e.g., the N-methylglucamine salt, meglumine iothalamate (e.g., CONRAY™ contrast agent)); a salt of acetrizoate (e.g., sodium acetrizoate such as UROKON™ contrast medium); a salt of 3-5-diiodo-4-pyridone-N-acetic acid (e.g., the diethanolamine salt, iodopyracet such as DIODRAST™ contrast medium); or a salt of ioxaglate (e.g., sodium meglumine ioxaglate such as HEXABRIX™ contrast medium). In certain variations, combinations of radiopaque contrast agents may be used. As an example, an aqueous solution of diatrizoate meglumine and diatrizoate sodium may be used in some instances. In some variations, nonionic radiopaque contrast agents may be used. For example, iohexol (e.g., OMNIPAQUE™ contrast agent) and iodixanol (e.g., VISIPAQUE™ contrast medium) may be used in aqueous solution.

The type of contrast agent that is used, as well as its concentration, may be selected based on any of a variety of different factors, such as the X-ray absorption properties of the radiopaque compound (e.g., determined in part by the number of iodine atoms), the irradiation scheme and image capture scheme to be used (e.g., the intensity of the X-ray irradiation used to form the image, the time duration of the X-ray acquisition, the type of detector used, and the degree of contrast desired), and/or other issues specific to the patient (e.g., kidney disease, or the presence of other systemic drugs). In some cases in which a significant amount of radiopaque contrast agent is desirable, a contrast agent filter may be used (e.g., to minimize kidney damage).

Worldwide, over 80 million doses of iodinated contrast media are administered each year, e.g., for angiography or computed tomography, corresponding to approximately 8 million liters. This is one of the highest volumes of medical drugs used (Katzberg (2006), Kidney Int Suppl 100: S3-7). Contrast medium-induced nephrotoxicity (CIN) remains one of the most clinically important complications associated with the administration of contrast media. It is a common cause of hospital acquired acute renal failure and may require dialysis. Therefore, the condition of contrast medium-induced nephrotoxicity is both dangerous and cost intensive. Patients experiencing contrast medium-induced nephrotoxicity generally have more complications, more serious long term outcomes and a prolonged hospital stay than patients without CIN resulting in increased medical cost (Aspelin, P. (2004), Nephrotoxicity and the Role of Contrast Media, Radiation Medicine, 22(6): 377-378).

The exact mechanism behind CIN is not completely understood. It was suggested that causes of CIN are renal ischemia (by reducing blood flow or increasing oxygen demand) and, presumably, direct toxicity of the contrast medium to tubular epithelial cells. Moreover, the osmolality and the viscosity of the contrast medium are thought to play a role in CIN. Moderate to severe chronic kidney disease (CKD) defined as estimated glomerular filtration rate <60 ml/min per 1.73 m2 (National Kidney foundation, CKD stages 3 and 4) is the most important risk factor of CIN. Therefore, these patients should be excluded from an examination that requires the administration of large doses of a contrast medium. Other major risk factor of CIN include older age, diabetes mellitus, intraarterial contrast medium administration, use of larger contrast medium doses, the concurrent use of nephrotoxic drugs, patient dehydration and any other condition associated with decreased effective circulation volume (Solomon, R. J. et al. (2007) Cardiac Angiography in Renally Impaired Patients. Circulation 112: 3189-3196). However, also patients who do not have an apparent renal disease or disorder are also at risk of suffering from CIN after the administration of a contrast medium.

To date, there is no optimal strategy to prevent CIN. Recent guidelines recommend intravenous volume expansion with a saline solution, use of low- or iso-osmolality contrast medium and limiting the volume of administered contrast medium (Solomon R., (2006) How to prevent contrast-induced nephrotoxicity and manage risk patients: Practical recommendations. Kidney Int 69: S51-S53). However, even when following these guidelines, there is still a significant risk of CIN after the administration of contrast media.

A cornerstone for reducing risk of CIN is to limit the total dose of contrast that a patient receives during an angiogram. For this reason, it is important to accurately determine the volume and dose of contrast material that is injected into the patient during a procedure. This is challenging since contrast material is frequently diluted with saline prior to injection and may be sent to a waste bag prior to injection. Medical personnel frequently make an “educated guess” of the amount of injected contrast based on such factors as dilution and residual fluids in the operating environment (e.g. waste, injector, syringes, etc.).

Various devices have been proposed to measure and potentially limit the total delivered volume of contrast media during an interventional procedure, but none has achieved widespread adoption. In a typical procedure, medical personnel have only a qualitative approximation of the amount of contrast that has been administered. Thus, there remains a need for an ability to monitor total delivered volume or concentration, and to provide quantitative feedback to the attending physician, so that the ongoing procedure can be conducted in a manner that takes into account the total delivered contrast media.

### SUMMARY OF THE INVENTION

There is provided in accordance with one aspect of the present invention, a system for measuring total delivered dose of contrast media during an intravascular procedure. As used herein, total delivered dose of contrast media can refer to either total delivered volume of contrast media or total delivered dose of an analyte of interest (e.g., a radiopaque agent such as an iodine containing salt) contained within the total volume of contrast media, each of which can be mathematically derived from the other. The system comprises an influent connector, configured to connect to at least one source of fluid and an effluent connector, configured to connect to an infusion port on a catheter. A flow rate detector is configured to measure the rate of fluid flow through the system, and an analyte detector is configured to detect the concentration of an analyte of interest flowing through the system. In certain applications, the analyte of interest comprises one or more iodine containing salts.

The flow rate detector may comprise a detector for determining change in refractive index, for determining thermal marker transit time, for determining change in electrical conductivity, or other techniques for determining flow. The analyte detector may comprise a light source and a sensor. The light source may comprise a source of UV light, such as a source of light suitable for evaluating absorption at about 244 nm.

The system may comprise an optical flow cell in fluid communication with the effluent connector, having the light source on a first side of the optical cell and the detector on an opposing side of the optical cell. At least one of the light source and the sensor may be attached to the optical cell. At least one of the light source and the sensor may be attached to a console, and the optical cell is configured for removable engagement with the console. In one implementation of the invention, the optical cell and associated tubing is configured for a single use. Each of the light source and the sensor may be attached to a console, spaced apart from each other to define a cavity to receive the one-time use optical cell.

The source of fluid may comprise a container of an infusate such as a saline bag, and at least one contrast injection port. The light source may comprise a light emitting diode. The sensor may comprise a CCD sensor, a CMOS sensor or other sensor having sensitivity corresponding to an absorption peak of interest.

The system may further comprise a memory, configured to store total delivered dose data from a plurality of discrete intravascular procedures, and one or more displays for displaying cumulative delivered dose or countdown progress towards a predetermined maximum desired delivered dose target.

There is provided in accordance with another aspect of the present invention, a method of monitoring total delivered dose of contrast media injection during an intravascular procedure. The method comprises the steps of providing a system for measuring total delivered dose of contrast media, the system comprising a flow rate detector, an analyte detector, an effluent port connected to an infusion port on the catheter, a fluid (e.g., saline) source and an injection port and tubing configured to merge fluid from the fluid source and the injection port into a common flow path which flows through the flow rate detector and analyte detector. Fluid is infused from the fluid source and at least one bolus of contrast media is injected through the injection port. The system determines the total, cumulative amount of contrast media delivered through the common flow path during the intravascular procedure.

The intravascular procedure may comprise an angiographic or endovascular procedure, such as an angioplasty, stent placement, thrombolytic procedure, embolization procedure, electrophysiology procedure or other endovascular procedure in the heart, brain or peripheral arteries. Alternatively, the procedure may comprise a heart valve replacement or heart valve repair procedure, or an abdominal aortic artery graft deployment procedure.

There is provided in accordance with a further aspect of the present invention, a dosimeter for determining cumulative delivered dose of an analyte of interest. The dosimeter comprises a manifold, having at least one influent connector for connection to at least one and typically two or more sources of fluid and merging incoming fluid streams into a common flow path. An effluent connector is in fluid communication with the manifold, for connection to an infusion port on a catheter. A side branch is configured to divert a known portion (e.g., percent of total flow) of fluid flowing through the common flow path which exits the manifold. A container is provided in fluid communication with the side branch, for receiving the diverted portion of fluid.

At least one analyte sensor is provided for sensing an indicium of concentration of an analyte of interest in the container. The analyte of interest may include any soluble or insoluble compound that increases absorption of X-rays, such as an iodine containing salt.

A light source may be spaced apart from an analyte sensor along an optical path through the container. The dosimeter may additionally comprise a sensor for determining total volume of liquid in the container. The volume sensor may comprise an ultrasound transducer, for reflecting an ultrasound signal off of the surface of the fluid to determine fluid height, from which the total fluid volume in the container can be determined. The total volume delivered to the patient can be calculated or approximated, knowing the percent of flow diverted at the side port. The light source and analyte sensor may be carried by the container. Alternatively, the light source and analyte sensor are carried by a console configured to removably receive the container. Further features and advantages of the present invention will become apparent to those of skill in the art from the detailed description of preferred embodiments which follows, when considered together with the attached drawings and claims.

### BRIEF DESCRIPTION OF THE DRAWINGS

FIG. 1 is a schematic flow diagram of a plurality of fluid sources directed through a dosimeter in accordance with the present invention and into the patient.

FIG. 2 is a schematic illustration of a dosimeter in accordance with the present invention.

FIG. 3 is a schematic illustration of a console, for removably receiving a detection module in accordance with the present invention.

FIG. 4 is a detection module configured to cooperate with the console of FIG. 3.

FIG. 5 is an optical flow cell set, having a plurality of different optical path lengths.

FIG. 6 is an optical flow cell, having a plurality of different optical path lengths.

FIG. 7 is a schematic illustration of a thermal marker transit time flow rate detector.

FIG. 8 is a schematic illustration of a conductivity flow rate detector.

FIG. 9 is a schematic view of an alternate thermal marker transit time flow rate detector.

FIG. 10 is a thermal transit time flow module configured to determine flow rate through a tube without invading the tube or contacting fluid within the tube.

FIG. 11 is a schematic view of an alternate thermal marker transit time flow detector.

FIG. 12 is a schematic view of a side branch sampling system and dosimeter.

FIG. 13 is a schematic view of an alternate side branch sampling system and dosimeter.

### DETAILED DESCRIPTION OF THE PREFERRED EMBODIMENT

The present invention provides a dosimeter for real time tracking and display of total delivered dose of a preselected component in a fluid stream. The dosimeter may additionally track and display the total delivered volume of fluid, such as the sum of the preselected component and a carrier such as saline. The dosimeter of the present invention will be discussed below primarily in the context of infusion of fluids into a patient, and, in particular, for monitoring the total delivered dose of contrast agent such as for facilitating fluoroscopy. However, the dosimeter of the present invention may be utilized for monitoring other fluids as will be apparent to those of skill in the art in view of the disclosure herein.

In a variety of interventional procedures a patient receives a flow of saline from a fluid flow line through a catheter and to a treatment site. The flow rate of the saline can fluctuate over time. In addition, a periodic bolus of contrast media can be mixed with the varying flow of saline and introduced into the flow line at various times and in varying concentrations. To monitor the total cumulative does of contrast and the total volume of saline delivered to the patient where the flow rates and doses vary over time, the present invention provides systems and methods to monitor the flow rate and contrast media concentration (e.g., by measuring absorbance of the solution) in the fluid flow line.

Measuring the flow rate of the solution in the fluid flow line can be accomplished using any number of suitable techniques discussed in greater detail below. One such technique includes introducing a thermal marker into the solution and measuring the temperature of the marker downstream. Other suitable techniques can include using a micro-flow meter, a mechanical flow meter, an optical flow meter, a pressure-based flow meter, or the like.

If the flow rate were known at all times, Q(t), the total amount of solution delivered to the patient, D_{TOTAL}, between times t₁ and t₂ would be given by the equation:

D_{TOTAL}=∫ₜ_{₁}^{t}^{²}Q(t)dt

However, in cases where the system acquires measurements of the flow rate at particular times, the integral equation can be approximated using a finite sum. For example, where the system acquires N measurements of the flow rate, Q′ᵢ, between times t₀ and t_{N-1}, the estimated total amount of solution delivered to the patient, D′_{TOTAL}, would be given by the equation:

$${D}_{TOTAL}^{′} = {\sum }_{i = 0}^{N - 1} {Q}_{i}^{′} ( {t}_{i + 1} - {t}_{i} )$$

The precision and accuracy of the estimate of the amount of delivered solution depends at least in part on the frequency of measurement of the flow rate, the accuracy and precision of the flow rate measurement value, and the variation in the flow rate. The first factor can depend at least in part on the method of measuring the flow rate and limitations imposed by the method. The second factor can depend on the instrumentation used in conjunction with the measurement method. The third factor may be outside of the control of the system, but understanding the potential variations (such as the magnitude of flow rate variations, the frequency of the variations, and the like) the first two factors can be adjusted to increase or optimize the quality of the estimate. For most situations, the flow rate is not constant but is delivered in bolus fashion through a syringe that is intermittently connected to the catheter. Flow rate is a secondary consideration but may be important if thermodilution is used.

Estimates of the concentration of the contrast media in the solution in the fluid flow line can be based on the absorbance, A, of the solution at a particular wavelength, λ. The absorbance of the solution can be defined as:

$${A}_{λ} = - {log}_{10} ( \frac{{I}_{1}}{{I}_{0}} )$$

where I₁ is the intensity of the light after passing through the solution and I₀ is the intensity of the light incident on the solution. Measuring the absorbance of the solution can be accomplished using any suitable technique. For example, the fluid flow can run through a single cell spectrophotometer which uses light at 244 nm to measure the absorbance. That wavelength corresponds to an absorbance maximum for iodixanol (e.g. Visipaque™) and Nycodenz®, known contrast agents. The system can be configured to measure the absorbance as a function of time.

The absorbance of the solution can be affected by the concentration of the contrast media in the solution. Accordingly, the absorbance, A_{λ}, can be expressed as a function, F, of the concentration of the contrast media, φ, in the solution:

A_{λ}=F(φ)

The function, F(φ), can be characterized for a range of concentrations in a saline solution through any suitable method. For example, measurements of the absorbance can be made for different measured concentrations of contrast media, φ′, and the results can be plotted and fit using a suitable function, F′(φ′). The absorbance estimate function, F′(φ′), thus represents an estimate of the absorbance of a saline solution with a measured concentration of contrast media, φ′.

The absorbance estimate function can then be used to generate an inverse function, F′⁻¹, which provides an estimate of the contrast media concentration, φ′, given a measured absorbance of the solution at a given wavelength, A′_{λ}:

φ′=F′⁻¹(A′_{λ})

An estimate of the concentration of contrast media in the solution as a function of time, φ′(t), can thus be obtained by measuring the absorbance of the solution as a function of time, A′_{λ}(t).

Similar to the calculation of the total amount of solution delivered to the patient, the total cumulative dose of the contrast media to the patient can be calculated based on the contrast media concentration, φ(t), and the flow rate, Q(t), of the solution. If these quantities were known, the total cumulative dose of contrast media, D_{contrast}, would be given by the equation:

D_{contrast}=∫ₜ_{₁}^{t}^{²}Q(t)φ(t)dt

Because these quantities are not known exactly, but rather monitored at discrete points in time, the estimated total cumulative dose of contrast media, D′_{contrast}, can be obtained using a finite sum. For example, where the flow rate and absorbance of the solution is measured at N discrete points in time, the estimated total cumulative dose of contrast media is given by the equation:

$${D}_{contrast}^{′} = {\sum }_{i = 0}^{N - 1} {Q}_{i}^{′} {F}^{′ - 1} ( {A}_{λ , i}^{′} ) ( {t}_{i + 1} - {t}_{i} )$$

where Q′ᵢ is the flow rate measured at time tᵢ and A′_{λ,i} is the absorbance measured at time tᵢ.

The accuracy and precision of the estimate of the total delivered dose of contrast media depends at least in part on the frequency of absorbance measurements, the accuracy and precision of the absorbance measurement value, and the variation in the concentration of the contrast media. The first factor can depend at least in part on the method of measuring the absorbance and limitations imposed by the method. The second factor can depend on the instrumentation used in conjunction with the measurement method. The third factor depends upon physician behavior and is outside of the control of the system, but understanding the potential variations in contrast media concentration (such as the quantity of contrast media per dose, the frequency of the doses, the duration of the doses, and the like) the first two factors can be adjusted to increase or optimize the quality of the estimate.

Using these equations, the estimated total amount of saline delivered to the patient, Dₛₐₗᵢₙₑ, can be monitored as well, using the equation:

D′ₛₐₗᵢₙₑ=D′_{TOTAL}−D′_{contrast}

The total cumulative dose of contrast media can be estimated and monitored using the techniques outlined above. In some instances, the equations and functions presented may depend on other variables that are not discussed. In many such cases, these dependencies can be discovered and corrected through calibration procedures. Similarly, appropriate constants may be required to transform each measured or estimated quantity into a quantity with appropriate units.

Other methods of implementing the techniques described above may be utilized as well. For example, rather than using functions to express relationships between variables, look-up tables can be used. As another example, numerical techniques can be used to estimate integrals rather than using the finite sums above. As another example, graphs such as a predetermined calibration curve can be used to represent the relationship between contrast medial concentration and absorbance, which may be advantageous in some instances where the resulting functional relationship between the variables is not readily invertible.

It is to be understood that the measure of absorbance can depend on the total amount of solution in the optical path of the light being used. One method to account for this dependence is to characterize the absorbance as a function of concentration using the same amount of solution in the optical path as there will be when the absorbance will be measured in situ. Another method to account for this is to generate a function which accounts for the amount of solution in the optical path.

In the description above, primes were used to indicate where a value was measured or estimated as contrasted with the actual or true value (e.g., estimated total dose of contrast media is expressed as D′_{contrast} whereas the actual total dose of contrast media is expressed as D_{contrast}.)

In general, the dosimeter of the present invention is configured to take measurements from which analyte concentration and fluid flow rate can be determined, and includes a console containing electronics, displays and controls. Sensors in communication with the console are preferable isolated from contact with the fluid stream being introduced into the patient. Alternatively, for sensors which contain any components in direct contact with the infused fluid, the portion of the device which comes into contact with infused fluid is preferably configured for one-time use, such that it may be disposed following treatment of the patient.

Referring to FIG. 1, there is illustrated a simplified flow diagram for the context of the present invention. A first source of fluid such as a syringe 10 and a second source of fluid such as an IV bag 14 are in communication with a patient. Additional fluids sources may be provided, as is understood in the art. The syringe 10 is in communication with a manifold 18 by way of flow path 12, and the IV bag 14 is in communication with the manifold 18 by way of a flow path 16. Manifold 18 may be a discrete structure, or simply a confluence of flow paths 12 and 16, downstream from which all flow paths are merged into a common flow path 20. Flow path 20 is directed via conventional IV tubing or other conduit directly to the patient 22. This may be accomplished, for example, by coupling a luer connector on the downstream end of common flow path 20 to a fluid infusion port on the proximal manifold of a catheter (not illustrated) through which fluids and contrast media is to be introduced.

The dosimeter 24 in accordance with the present invention is positioned along the common flow path 20, downstream from the merger of various fluid introduction ports at manifold 18, and upstream from the patient 22. Thus, all fluid entering the patient via the catheter, from whatever source, travels through the dosimeter 24.

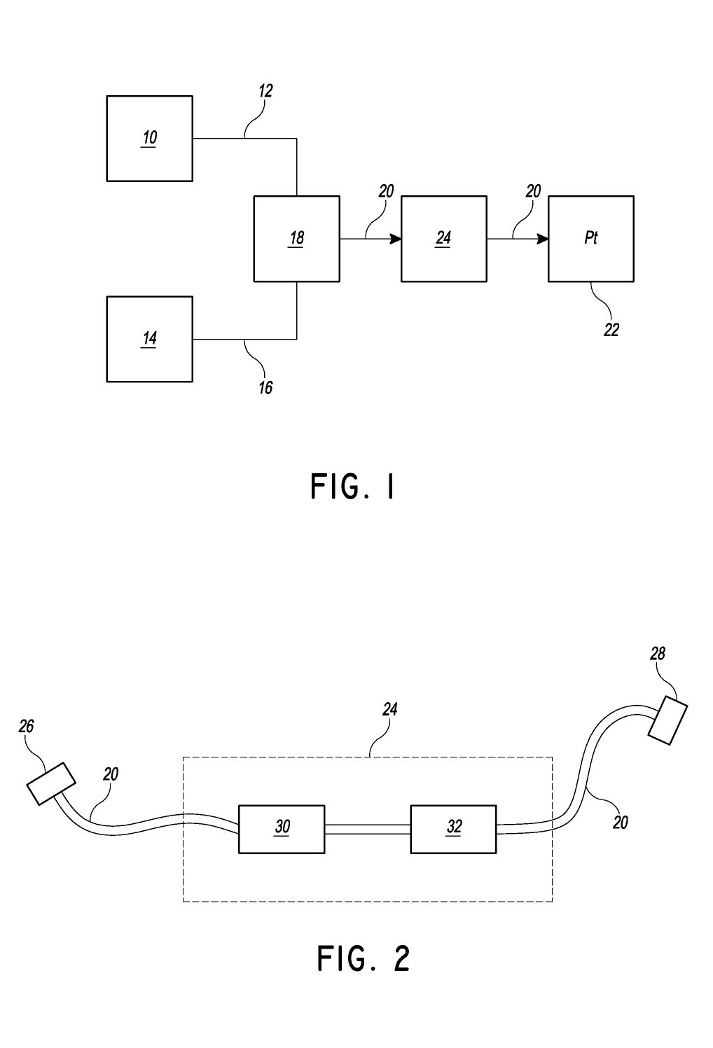

Referring to FIG. 2, the dosimeter 24 includes a proximal connector 26 such as a luer connector for connection to the manifold 18 or elsewhere along the common flow path 20. Dosimeter 24 additionally includes an effluent connector 28, such as a luer connector for connection to a proximal infusion port on a catheter. Fluid flowing from proximal connector 26 through common flow path 20 and into the patient passes through a flow module 30 and a detection module 32. The flow module 30 is configured to determine an indicator of the rate of fluid flow through common flow path 20. The detection module 32 is configured to determine the amount of contrast media or other analyte of interest that flows through the common flow path 20 and into the patient. The flow module 30 and detection module 32 may be coupled in series flow or parallel flow relationship to each other, and may be housed in a common housing, or separate housings, spaced anywhere along the length of the common flow path 20 between the manifold and the patient. Additional details of the flow module 30 and detection module 32 will be provided below.

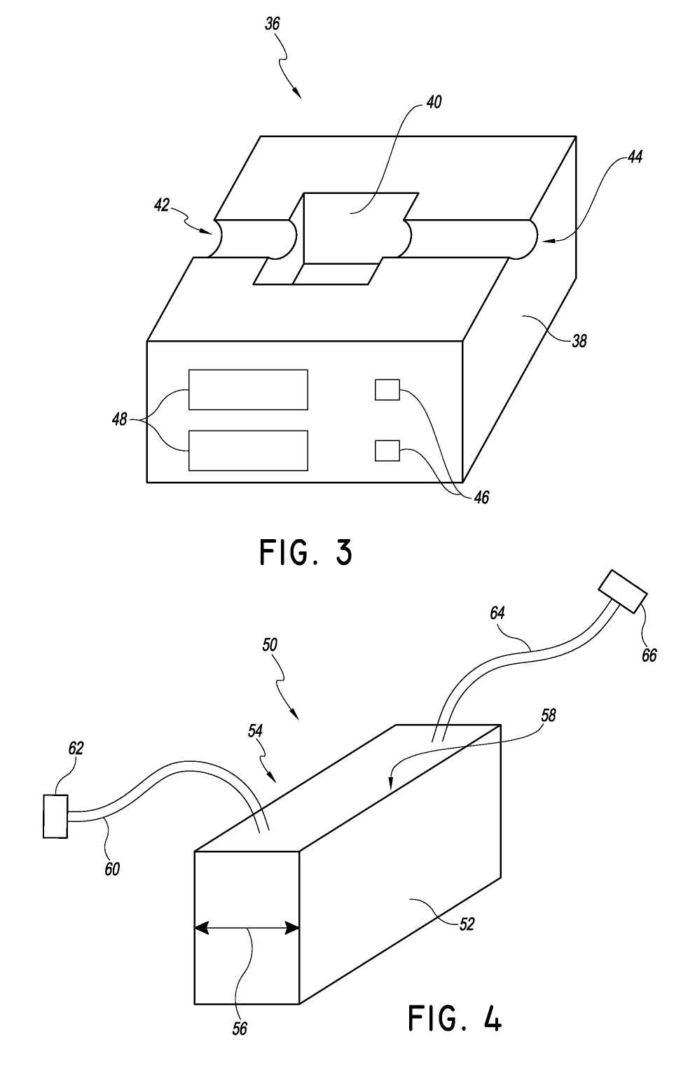

Referring to FIG. 3, there is schematically illustrated a console 36 in accordance with one implementation of the present invention. Console 36 includes a housing 38, containing a chamber 40 for receiving at least a component of the dosimeter 24. In one implementation discussed in greater detail below, the chamber 40 is configured to removeably receive a disposable optical flow cell for spectrophotometric analysis.

The housing 38 may additionally be provided with influent flow path or tubing guide 42, and effluent flow path or tubing guide 44. A cover (not illustrated) may be provided, such as to hingeably close over a dosimeter component positioned within the chamber, during operation.

The influent guide 42 or effluent guide 44 or both may be provided with fluid flow detectors as will be discussed in greater detail below. Alternatively, the fluid flow detector may be positioned elsewhere along the common flow path 20 between the manifold 18 and the patient 22.

Housing 38 may additionally be provided with any of a variety of controls 46, such as on/off controls, display mode controls, and other depending upon the desired functionality of the system. One or two or more displays 48 may be provided on the housing 38 or in electrical communication with the housing, for displaying information about the fluid stream. Displayed information may include the total cumulative delivered volume or dose of contrast media or other analyte of interest. Total delivered fluid may additionally be displayed. Other information about the infused fluid, such as pH, temperature, delivery rate, or the presence and/or amount of a second or third analyte of interest may be displayed. Display 48 may be provided on the housing 38, or remote from the housing 38, via wired or wireless connection such as on an iPad, notebook computer, or other monitor, attached to a wall, carried by a support arm or suspended from the ceiling, or elsewhere as convenient depending upon the configuration of the cath lab or other medical facility.

One method for measuring contrast media or other analyte in accordance with the present invention is by monitoring one or two or more wavelengths at which the absorbance spectrum for the analyte has a characteristic that correlates with concentration. This may be accomplished, for example, by measuring absorbance at a preselected wavelength through a spectrophotometer optical flow cell 50. See FIG. 4. The flow cell 50 comprises a first window 52 and a second window 54 separated by a predetermined optical path length 56. The composition of the windows 52 and 54 may be varied depending upon the selected wavelength of the analysis. For example, silica maybe used in the ultraviolet range, and quartz may be used in the far ultraviolet and near infrared range as it is known in the art.

The optical path length 56 and sample volume of the chamber may be optimized for each particular analysis depending upon variables such as the anticipated optical density range of the analyte. Optical path lengths 56 in the vicinity of 0.5 mm up to 10 mm or more may be useful in many ultraviolet or visible range analyses. One contrast agent of interest, discussed more below, absorbs strongly in the UV with an absorbance maximum of about 244 nm. Depending upon the analyte concentration, a standard 1 cm path length quartz cell in a single beam spectrophotometer may be desirable.

The flow cell 50 defines a chamber 58 positioned between the windows 52 and 54. Chamber 58 is in fluid communication with an inflow line 60, having a connector 62. Connector 62 may be a luer connector or other standard fitting for connection to a manifold 18 or common flow path 20 through which all infused fluid must pass.

The chamber 58 is also in fluid communication with an effluent line 64. Effluent line 64 is provided with a connector 66, such as a luer connector, for connection to the infusion port on the catheter.

By connecting effluent luer 66 to the influent port on the catheter, all fluids which will enter the patient via the catheter must flow through the flow cell 50.

The flow cell 50 is intended for a single use after which is to be disposed. In use, the flow cell 50 is connected to the catheter and to the manifold as described above, and inserted into the chamber 40 of the detection module 32. A source of ultraviolet light is provided on a first side of the flow cell 50, and a detector such as a CCD or CMOS detector is provided on a second side of the flow cell 50. In this manner, absorbance of the solution flowing through the flow cell 50 can be measured in accordance with techniques well understood in the spectrophotometry arts.

The optimal wavelength or set of two more wavelengths can be determined by comparing the absorbance spectrum of the analyte of interest (e.g., the contrast media) with the absorbance spectra of the background solution (e.g. Saline).

The detection module 32 may additionally be provided with a dilution capability, to quantitatively add diluent (e.g., saline) to the inflow line 20 if necessary to bring analyte concentration into the working range. Alternatively, if the absorbance is measured at a different (e.g. higher) wavelength, dilution may not be required. The desirability of dilution, selection of ideal wavelength, and other variables can be determined by those of skill in the art through routine experimentation for each desired analyte, in view of the disclosure herein.

Typically, concentration of an analyte can only be quantitatively correlated to absorption over a working range. When the concentration of the analyte in a carrier fluid such as saline exceeds the high end of working range, conventional laboratory practice is to quantitatively dilute the solution to bring the analyte concentration back into the working range. If analyte concentration becomes too low, concentration steps may be accomplished, or the optical path length of the flow cell may be increased. In the context of the present invention, the concentration of contrast media may range from 0 (most of the time) to relatively high as a bolus of contrast is injected to enhance visualization. The effective working range of the dosimeter must therefore be quite large, and able to work in real time preferably without dilution steps. This may be accomplished in several different ways.

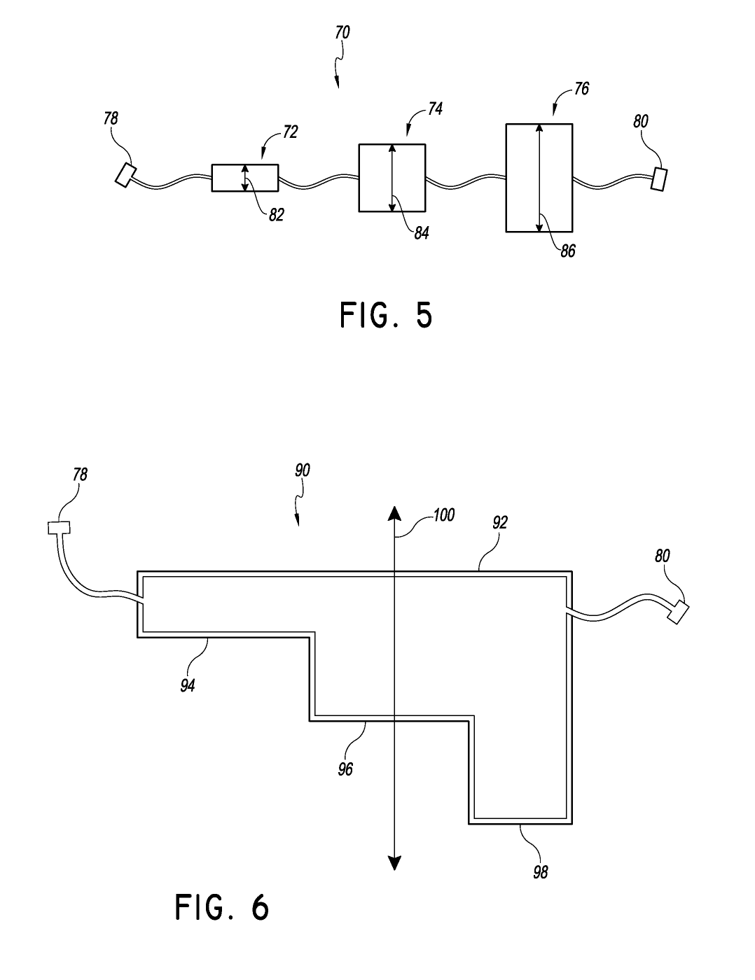

Referring to FIG. 5, there is disclosed a flow cell set 70 for use as an alternative to flow cell 50 illustrated in FIG. 4. Flow cell set 70 comprises a first flow cell 72, at least a second flow cell 74, and, in the illustrated implementation, a third flow cell 76. A fourth or fifth or additional flow cells may additionally be included in the flow cell set 70. All of the flow cells 72, 74 and 76 are in fluid communication with an influent port 78 and an effluent port 80. Flow cells 72, 74 and 76 may be in parallel flow or series flow communication.

Flow cell 72 has a first optical path length 82. Second flow cell 74 has a second, greater optical path length 84, and third flow cell 76 has a third, greater optical path length 86. The flow cell set can be physically configured to drop into a chamber or a series of chambers on a console 36 such that a light beam is directed along the optical path of each of the flow cells. The light beam may be generated by a unique light source such as an LED for each flow cell, or a common light source potentially in the nature of a split beam spectrophotometer.

The path length for each of the flow cells can be determined through experimentation by those of skill in the art in view of the wavelength selected and the analyte of interest. For example, path length 82 may be no more than about 1 cm or no more than about 0.5 cm. Path length 84 may be between about 0.5 cm and about 1.5 cm or 2.0 cm. Path length 86 may be between about 1.5 cm or 2 cm and 5 cm or greater. When all three flow cells are filled with fluid and absorption at the selected wavelength is determined, software can be configured to identify which flow cell is appropriate to bring the analyte concentration within the working range and absorbance input from the other two or more flow cells is simply disregarded until the analyte concentration changes sufficiently that a different path length is required to keep analyte concentration within a working range. This allows continuous real time monitoring over significant variations in analyte concentration.

The foregoing can alternatively be accomplished in a single customized flow cell 90. See FIG. 5. Flow cell 90 includes a first window 92, and a set of second windows 94, 96 and 98 parallel to the first window 92 and each defining a unique optical path parallel to optical axis 100.

An alternative way to effectively extend the operating range of the spectrophotometer is to measure absorbance at more than one peak that is unique to the analyte of interest. When analyte concentration becomes outside of the working range at a first absorbance peak, software can be configured to calculate concentration based upon absorbance at a second, different absorbance peak. This requires the presence of an appropriate absorbance spectrum, and might not work well with contrast media having only a single dominant absorption maximum.

For this purpose, one or more dyes may be introduced into the contrast media at known concentrations, to create one or two or three or four or more absorbance peaks at desired points within the UV, visible or infrared spectrum, which can be simultaneously monitored by the spectrophotometric detector. Software can be configured to select the wavelength appropriate for the transient analyte concentration, for use in computing total delivered dose.

Dosimeter 24 is additionally provided with a flow module 30 for determining the fluid flow rate into the patient, and correlating transient flow rate data in time with absorbance data to allow calculation of total cumulative delivered dose. The flow module 30 may be a part of the disposable component and placed in fluid communication with the flow cell 10. Alternatively, the flow module may be formed as a portion of the reusable console 36.

In a typical interventional procedure, such as an angioplasty, angiogram, abdominal aortic aneurysm graft deployment, percutaneous heart valve repair or replacement, neuro embolectomy, for example, the influent flow via inflow line 20 will variably include saline, periodic drug, and periodic bolus of contrast media. Thus, calculations to determine total delivered dose of contrast media must take into account fluctuations in absorbency over time as a function of fluid flow.

The fluid flow rate flowing through common flow path 20 may be determined in any of a variety of ways, depending upon desired performance and complexity of the system. In general, flow rate is preferably determined in a manner that does not require bringing the influent flow into direct contact with any kind of energy source or detectors. Thus, the energy source or detectors preferably are able to function to determine flow rate through the wall of the IV tubing or other window or sealed membrane which prevents contamination of the influent fluid. Depending upon the analyte of interest, fluid flow measuring techniques include measuring a variable or marker that is indicative of flow, such as measuring change in refractive index, thermal marker transit time, and changes in conductivity which can be correlated to analyte concentration. Some of these techniques will be outlined below.

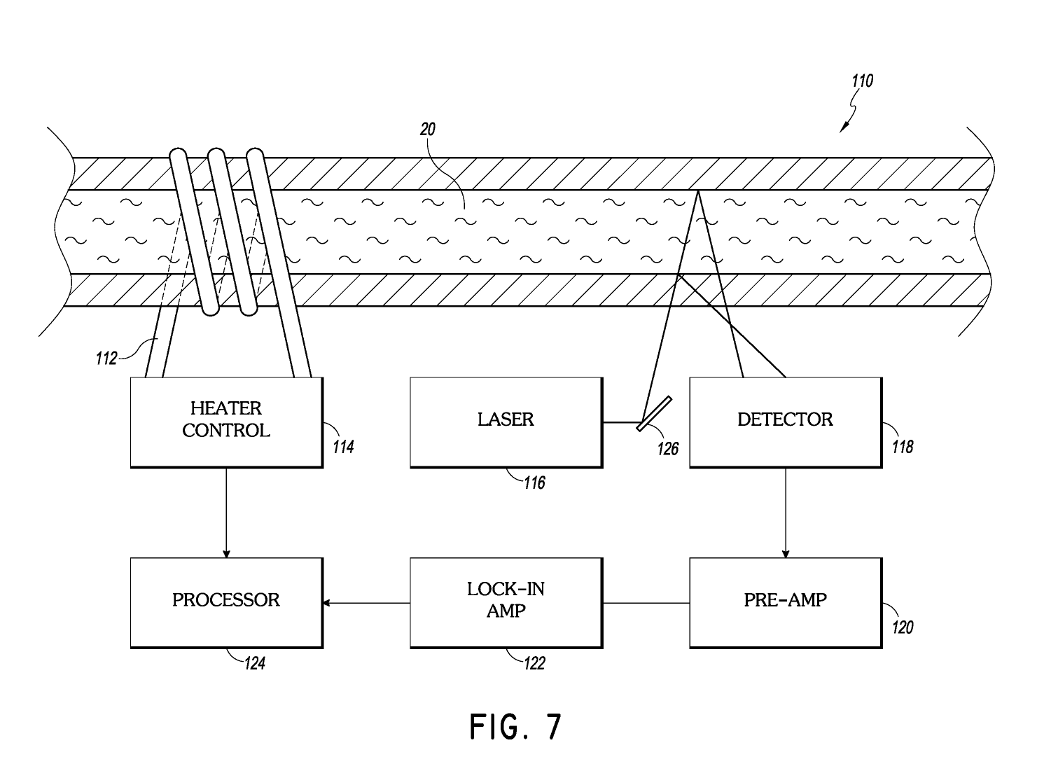

With reference to FIG. 7, an isolated (i.e., no contact with infused fluid) system 110 for determining flow rate within a fluid-bearing passageway such as common flow path 20 monitors the refractive index of the fluid within the passageway in order to obtain data for calculating flow rate. The interdependency between temperature and refractive index for certain analytes and fluids is recognized in the art.

The component arrangement of FIG. 7 is merely exemplary, and other components may be utilized. The system 110 includes a heat source such as a heater 112 and a heater control device 114. The heater control device may energize the heater in a fixed repeating pattern (e.g., a sine wave pattern). A laser 116 introduces an interrogation beam, which is reflected at the “forward” and “rearward” interfaces of the fluid with the wall of the passageway 20. A detector 118 senses an interference pattern which is dependent upon the temperature of the fluid within the passageway. The output of the detector is amplified by the preamplifier 120. The lock-in amplifier 122 cooperates with the processor 124 to monitor the phase difference between the fixed repeating pattern defined by the heater control device 114 and the changes in the interference pattern, as sensed by the detector 118. In one embodiment, the phase difference is used to identify the flow rate of the fluid.

The heat source may be an electrically conductive coil, LED, laser, ultrasound transducer, thermal conductivity element or a serpentine pattern of traces through which current is conducted in order to generate heat in the area of the passageway 20. The heater is activated in a known pattern in order to introduce heat tracers into the flowing fluid within the passageway 20. For example, sine wave or square wave activation may be employed. In the embodiment of FIG. 7, the transit time for passage of a heat tracer from the region of the heater to an interrogation region is determined in order to identify the flow rate. However, the heater may be located within the interrogation region. As will be described more fully below, the thermal dilution approach may also be used.

The thermal energy that is introduced to the fluid by the heater 112 should be sufficiently great that the change in temperature of the fluid is perceivable using the techniques to be described below. However, the increase in temperature should not be high enough to affect the chemical composition of the fluid. Optionally, the system may include a feedback loop to the heater controller 114 to provide active temperature control.

The illustrated system 110 includes the laser 116. Preferably, the laser 116 is a “non-thermal” laser, i.e., one that generates light at a frequency which is not readily absorbed by the flow of fluid. For example, the laser may be a diode laser having a center frequency that is below 1100 nm. A helium-neon laser beam may be reflected by the mirror 126 toward a window within the fluid-filled passageway 12. One portion of the beam will be reflected at the “forward” interface of the fluid and the wall of the passageway. A second portion will be reflected at the “rearward” interface of the fluid and the passageway. As a result of constructive and destructive interference, a forward-scattered or back-scattered interference pattern will be generated and monitored by the detector 118. As is known in the art, the interference pattern is a series of maxima and minima in an intensity modulated beam profile that emerges radially from the passageway. Interference patterns are currently used in the industry and are described in an article entitled “Capillary-Scale Refractive Index Detection by Interferometric Backscatter” (Analytical Chemistry, Vol. 68, No. 10, May 15, 1996, pages 1762-1770), by H. J. Tarigan et al.

Typically, the beam of laser light is introduced at a non-right angle to the axis of the fluid-bearing passageway 20. In the embodiment of FIG. 7, the detector is a multi-element device, such as a charge coupled device (CCD). When light enters the fluid at the forward fluid-to-passageway interface, the light will be redirected, since there is a refractive index mismatch between the fluid and the structure that forms the passageway. The refractive index of the fluid will change with changes in the temperature of the fluid. As a result, the interference pattern will shift in position with changes in temperature. The direction of the shift will depend upon the direction of change of the refractive index. Consequently, the characteristics of the interference pattern that is received at the detector 118 will vary with fluctuations in the temperature of the fluid. The output signal from the detector 118 is received by the preamplifier 120. In one embodiment, the preamplifier provides signal amplification and converts the amplified signal to a voltage output, if the output of the detector 118 is an impedance change signal. Optionally, the system 110 includes the lock-in amplifier 122. The lock-in amplifier may be used to improve the performance of operations for monitoring the phase difference between the introduction of the heat tracers and the subsequent changes in refractive index.

In operation, the heat control device 114 activates the heater 112 to introduce a heat tracer (or a repeating pattern of heat tracers) into the fluid within the passageway 20. The heat tracer will flow with the fluid toward an interrogation region defined by the laser 116 and the detector 118. When the heat tracer reaches the interrogation region, the refractive index of the fluid within the region will be perceptibly different than the refractive index of fluid that has not been heated. The output of the detector 118 will change to reflect the shift. The signal from the detector is processed using the preamplifier 120 and the lock-in amplifier 122. The processor 124 receives signals from the lock-in amplifier 122 and the heater control device 114, so that it is able to identify the transit time of the heat tracer from the heater 112 to the interrogation region. The distance that the heat tracer travels is fixed, so that the flow rate can be determined using conventional techniques.

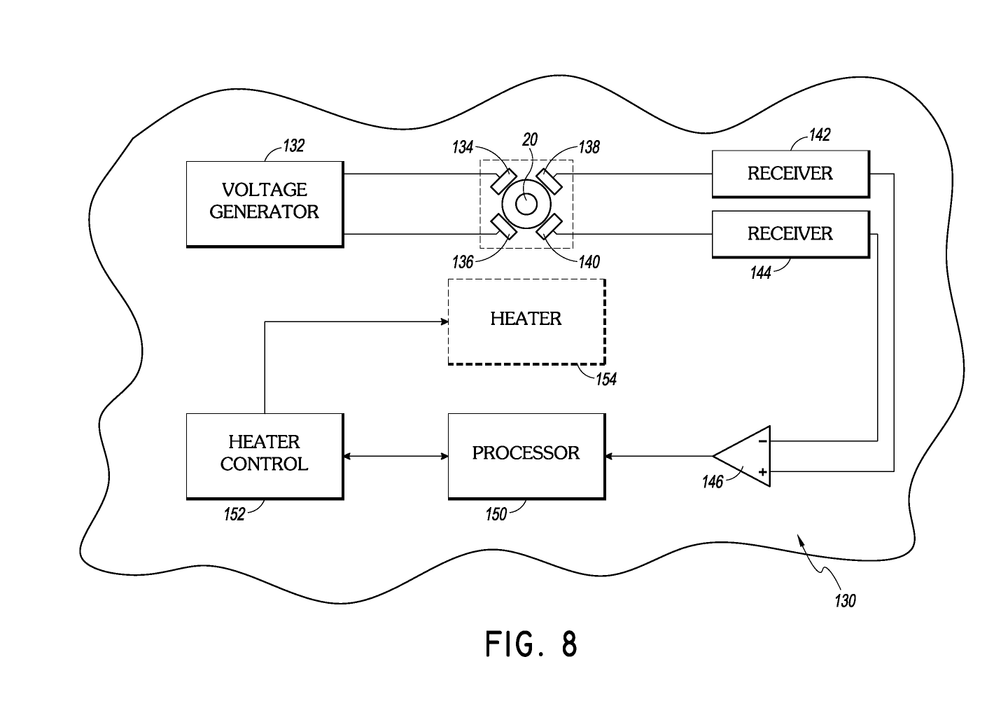

Rather than monitoring the refractive index of the fluid in order to determine when a heat tracer reaches an interrogation region, the conductivity of the fluid may be monitored. An embodiment for determining flow rate based upon fluid conductivity is shown in FIG. 8. The system 130 includes a flow passageway 20, as has been discussed. A removable disposable detector cell 131 is provided, containing two or electrodes 70, 72, 74 and 76, in electrical communication with the fluid flowing through passageway 20. The detector cell 131 may be configured as a one-time use disposable element, having an influent port in communication with an effluent port through a central chamber. Influent port and effluent ports may be provided with luer connectors or other convenient connection, for positioning the detector cell 131 to form a portion of the flow passageway 20. The two or more electrodes described herein are positioned on the detector cell 131, in electrical communication with fluid flow travelling therethrough. Wires or other electrical connectors can place the detector cell 131 in electrical communication with suitable electronics contained in console 36 or other electrical control system.

Conductivity detection has been used in the art to detect specific ionic constituents within a liquid of interest. B. Gas et al. describe an arrangement for detecting zones of ionic species that are propagating through a capillary. “High-Frequency Contactless Conductivity Detection in Isotachophoresis,” Journal of Chromatography, 192 (1980), pages 253-257. The system of FIG. 8 is consistent with the arrangement described by B. Gas et al. However, other arrangements may be utilized, depending upon the availability of an analyte having conductivity changes which correlate to concentration, without diverging from the flow rate monitoring of the present invention.

A voltage generator 132 is connected to two of the electrodes 134 and 136. The frequency of the voltage generator may be one megahertz, but this is not critical. The generator provides signals to the electrodes 134 and 136 that are 180° out of phase. Thus, at least one capacitive cell is formed. In a less complex two-electrode system, an electrical diagram could include an AC (alternating current) source connected across two capacitors that are connected in series at opposite ends of a resistor. The capacitors represent the walls of the flow path and the resistor is the fluid within the flow path.

The electrodes 138 and 140 are separately connected to receivers 142 and 144. The receivers typically include preamplification circuitry. Signals from the voltage generator are capacitively coupled through the passageway 20 and are detected by the receivers 142 and 144. The outputs from the receivers are responsive to the strengths of the signals received at the electrodes 138 and 140. In the application illustrated in FIG. 8, the output signals from the receivers are connected to a differential amplifier 146. The signal from the differential amplifier 146 is responsive to the difference between the strengths of signals from the receivers, since the inputs to the electrodes 134 and 136 are 180° out of phase. However, use of a differential amplifier is not critical to the invention. Rather than coupling the receivers to a differential amplifier, the outputs from the receivers may be connected to an adder that may provide additional sensitivity to fluctuations in the conductivity of fluid within the passageway 20.

The differential amplifier 146 is connected to a processor 150, which is also in communication with a heater control device 152. The heater control device is used to activate and deactivate a heater 154 that is in thermal communication with the passageway 68. Since the heater is preferably (but not necessarily) located at a position along the passageway that is different than the interrogation region defined by the positions of the electrodes, the heater is shown in phantom.

Prior to activation of the heater 154, the fluid within the passageway 20 will have a particular level of conductivity. The conductivity will be dependent upon the ionic species that are constituent to the fluid of interest and upon any electrolyte that is used. The heater control device 152 may provide a notification signal to the processor 150 that the heater 154 has been activated to introduce a heat tracer, so that the processor is able to anticipate the arrival of the heat tracer at the interrogation region defined by the electrodes. This notification technique enables the processing circuitry to distinguish species-dependent conductivity fluctuations from the anticipated thermal-dependent conductivity fluctuation. More complex techniques may be substituted.

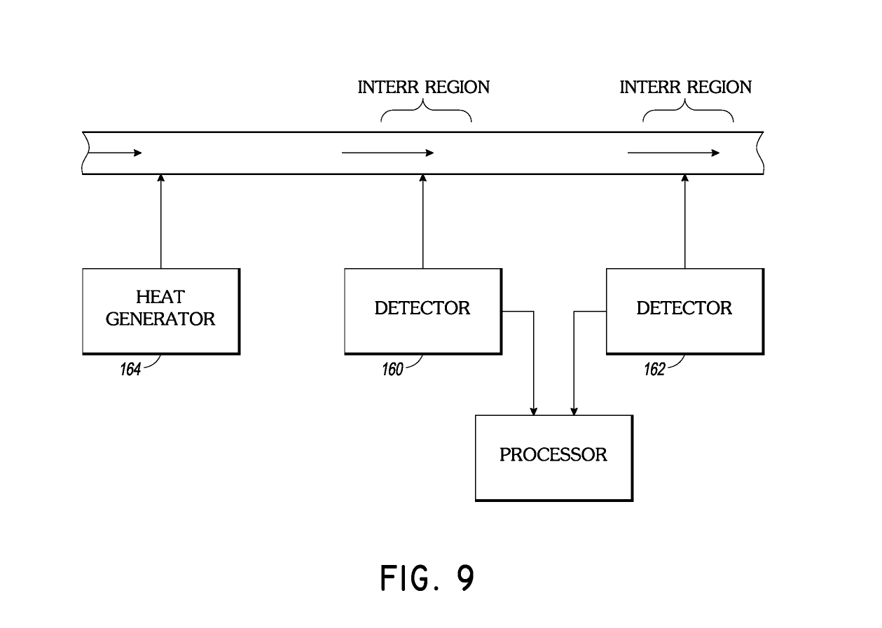

As an alternative to the cross-channel capacitively coupled embodiment of FIG. 8, conductivity fluctuations within the passageway 20 may be determined by capacitively coupling electrodes along the length of the passageway. Referring briefly to FIG. 9, the electrodes may be rings that are positioned at the interrogation regions of the two detectors 160 and 162. However, rather than two interrogation regions, there would be a single interrogation region along the lengthwise portion of the passageway between the two ring-shaped electrodes. When a heat tracer reaches the lengthwise interrogation region, the conductivity within this region will increase. Using the signaling techniques described with reference to FIG. 8, the detection of the heat tracer within the interrogation region may be used to determine the flow rate within the passageway.

Turning now to FIG. 9, a second detector 162 may be used to monitor flow rate between two interrogation regions, rather than monitoring flow rate from the position of the heat generator 164 to the first interrogation region defined by the detector 160. The first detector 160 will sense the arrival of the heat tracer at a region that is downstream of the heat generator, but upstream of the interrogation region of the second detector 162. The heat tracer will pass through the first interrogation region and will subsequently be detected by the second detector. The time necessary for propagation of the heat tracer between the two interrogation regions may be used to calculate the flow rate over the fixed distance between the two interrogation regions. For a fixed modulation of the heat generator 164, the phase difference between the two detections may be used to determine the flow rate.

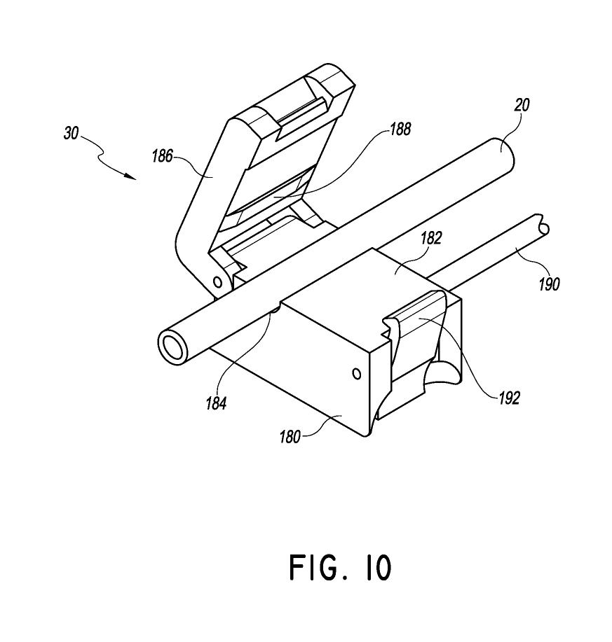

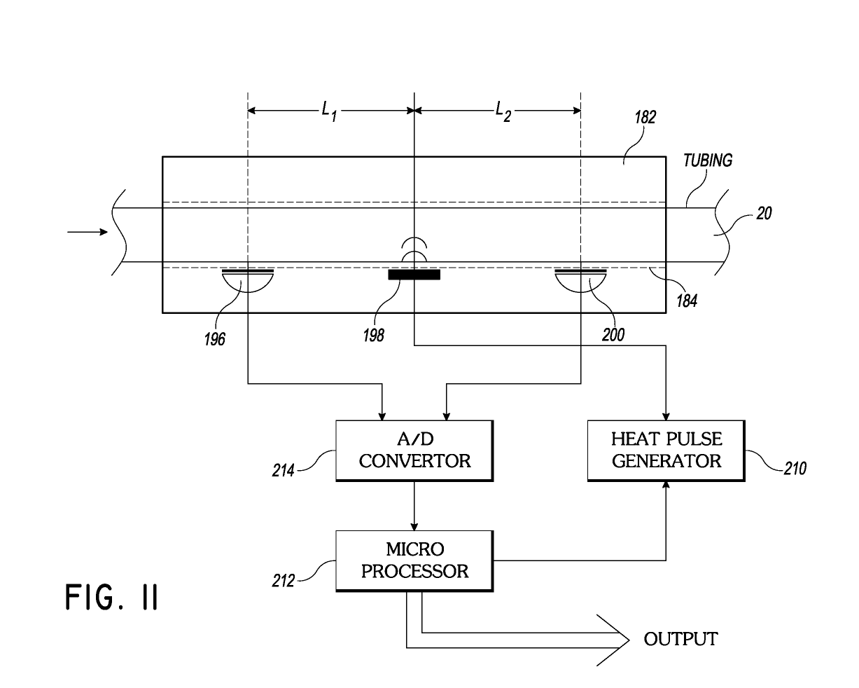

A simple, isolated thermal transit time flow module 30, having thermal correction, is illustrated in FIG. 10. Flow module 30 may be integrated into the console 36, as has been discussed, or placed elsewhere along the fluid flow path 20.

FIG. 10 shows a housing 180 that can be made of any suitable material, such as plastic. The housing 180 includes a base 182 having a slot 184 that may be generally semi-circular in shape, and that extends across the base width. The slot 184 removably holds a tube 20 such as conventional IV tubing, in which the infused liquid flows.

Housing 180 as shown has a hinged cover 186 that has a slot 188 across its width to overlie the tube. A cable 190 extends from the base 182. The cable has the necessary wires to connect the temperature sensors and heating elements that are located in the base to circuitry that is used in providing power to the components and for measuring the flow rate. The cover 186 may have a latching mechanism 192 that holds the cover to the base 182. When the cover 186 is closed the tube 20 is held between the slots 184 and 188. The housing 180 and slots 184 and 188 can of any desired size and shape to accommodate the type and size of the tube in which the liquid flow rate is being measured.

FIG. 11 shows details of the base part 182 of the housing 180 in which a tube 20 is placed in the slot 184 with liquid flowing in the tube from left (upstream) to right (downstream), as shown in the drawing. Going from the upstream (source) direction of the liquid flow in the tube there are a first temperature sensor 196, a heating element 198 and a second temperature sensor 200. These components are described in detail below. The components 196, 198 and 200 are in the wall of the part of the base 182 in which the slot 184 is formed. The components can be molded into the wall if the base is molded or inserted into cavities made in the base to hold the components in thermal communication (e.g. contact with) the tube 20 when the lid 186 is closed.

The second temperature sensor 200 may be spaced from the heating element 198 by a known fixed distance designated as “L₂” which is a factor used in computing the liquid flow rate. The spacing between the first temperature sensor 196 and the heating element 198 may also be made the distance “L₁” which may be equal to L₂ for convenience in computation.

In one embodiment of the invention, the temperature sensors 196 and 200 are infrared (IR) IR heat detectors. Suitable IR heat detectors for use are Melexis—series MLX90614 obtained from Melexis, Inc. of Concord N.H. These IR detectors have a programmable response time, small size and are of relatively low cost.

A heat pulse generator 210 that may be external to the sensor base 182 supplies the required power to the heating element 198 to generate a pulse of energy to be transmitted through the wall of tube 20 to be applied to the liquid to heat it and form a heat marker. The timing of the application and the duration of the heat pulses is controlled by a microprocessor 212. The heat pulse generator and microprocessor, as well as all other electronic components can be within or external of the housing 180 as desired.

Alternative types of heating elements may be an ultrasonic transducer that receives voltage from the heat pulse generator 210 and converts the voltage into electro-mechanical (ultrasonic) energy. The US transducer preferably is of the type whose output energy can be focused to concentrate the energy at a fixed point in the liquid flowing in the tube. The energy pulse from the US transducer passes though the tube wall, and is absorbed by the liquid to produce a heat bolus, or mass, that serves as the heat marker. The US transducer would normally engage the wall of the tube 20 and would have sufficient power supplied by the generator with the power requirements being determined by the type of tube material and the tube wall thickness. Different types of liquids have different heat absorption factors to different ultrasonic energy frequencies. Therefore, the frequency of the ultrasonic energy is selected so that the maximum amount of heat will be absorbed by the liquid in the tube.

The heating element may alternatively be a laser diode that is suitably powered by the heat pulse generator 210. When a laser diode is used, the laser wavelength output can be selected to maximize the heat absorption by the liquid. In one embodiment of the invention, a laser diode is used having an output near about 1550 nm wave length. At this wave length the heat absorption coefficient of water and many other liquids is relatively high. Such a laser diode is relatively inexpensive and is commercially available. See, for example, Newport Corporation Spectra Physics Division (Santa Clara, Calif.) Model ML 925B45F. The light output energy from the laser diode can be focused directly from the diode or through an optical system to be concentrated for application into a selected point of the flowing liquid through common flow path 20.

In the operation of the system the ambient, or normal, temperature of the liquid is measured by the first temperature sensor 196. The heat marker in the liquid is sensed as it flows past the second temperature sensor 200. The liquid to be infused flows past the first temperature sensor 196 at the time t0. At time t1 a heat pulse marker H is applied to the liquid in the tube as explained above. The heat marker H then flows past and is detected by the second temperature sensor 200 at time t2. The second temperature sensor 200 is located at the fixed distance L from the heating element 109.

Measurement of the time of transit of the heat marker H over the fixed distance L gives the liquid flow rate in accordance with the following:

Q=AₓL/t_{d} where

Q=Flow rate

A=Cross sectional area of the tube

L=Distance between heating element 40 and temperature sensor 50

t_{d}=average transit time less the time lost due to the response of tube material in the heat detector. That is:

t_{d}=tₘ−tₜ−tₗ where

tₘ multiple time measurements

tt=Calculated delay in tubing due to thermal time constant associated with plastic tubing

tl=Response time of heat detector.

The time t_{d} is known in advance and is programmed into the microprocessor 212. Since all of tm, tt and tl are known the value td is calculated. Since A and L also are known, the flow rate Q is calculated by the microprocessor 212.

In the components of the electronic circuit, as shown in FIG. 2, the microprocessor 212 is programmed with the values A, D, tₜ and tₗ The outputs of the temperature sensors 196 and 200 are connected to an analog to digital (A/D) converter 214 that converts the measured temperature into digital format. Some temperature sensors include this function so that the A/D converter might not be needed. The microprocessor 212 produces a timing signal to cause the heat pulse energy generator 210 to produce an output that is applied to the heating element 198. The timing signal also starts a transit time period, compensated by the various delay factors discussed above, that is ended by the detection of the heat pulse by the second temperature sensor 200. The microprocessor calculates the flow rate Q from the measured transit time period using the formulas discussed above. The measured flow rate calculated by the microprocessor can be of any required dimensional quantity, e.g. cc/min, cc/hr or any other unit. This is the microprocessor output which can be displayed by a suitable display device 48 located on the housing 180 or 38 or output to a display remote from the housing as has been discussed. The calculated flow rate data can be supplied from the microprocessor output to another device to be used for flow rate control or any other purpose.

The microprocessor 212 is preferably programmed to make multiple measurements of the transit time td of the heat pulse from the heating element 198 to the second temperature sensor 300 and from these multiple measurements calculate the value tm. The microprocessor also can be programmed to perform as many calculations of Q over a predetermined period of time as desired, to average the calculations of Q, to take a maximum or some other value of Q from a group of measurements, etc.

Using the two temperature sensors 196 and 200 has an advantage in that common mode temperature changes can be eliminated. That is, the ambient (before heat pulse is applied) temperature of the liquid is measured by the first temperature sensor 196 and is used as a base line value by the microprocessor. The microprocessor 212 is programmed to respond to detection of a heat pulse marker H at a predetermined temperature above the base line value. Therefore, if the ambient temperature of the liquid varies either up or down it will have no effect on the accuracy of the flow rate measurement since the base line value varies in this manner. The same advantageous effect is obtained if a different liquid having a different ambient temperature is substituted.

Additional details and considerations in the construction of a fluid flow rate measurement are disclosed in U.S. Pat. No. 6,386,050 to Yin et al., entitled Non-Invasive Fluid Flow Sensing based on injected heat tracers and indirect temperature monitoring, and U.S. Pat. No. 7,908,931 to Dam, entitled Non-Invasive Flow Rate Measuring System and Method, the entire contents of which are incorporated by reference herein.

In accordance with a further aspect of the present invention, there is provided a real-time or intermittent side branch sampling system for determining the delivered dose of contrast media or other analyte of interest. In the side branch embodiment, a low flow branch line bleeds a small volume of fluid via a small “T” connector inserted into the injection stream such as in fluid flow communication at the injection luer on the procedure catheter. The branch line carries a low flow sample of fluid being injected into the patient, into a detection system. The detection system may comprise a real time monitor, such as a spectrophotometric detector as has been discussed. Alternatively, the side branch flow may be diverted into a removable container, such as a collection cell, which can be detached periodically from the system. The cell may be carried to a remote location in the same room or elsewhere, and processed to elute and determine a concentration of analyte, which can then be mathematically processed to determine the delivered dose of the analyte to the patient.

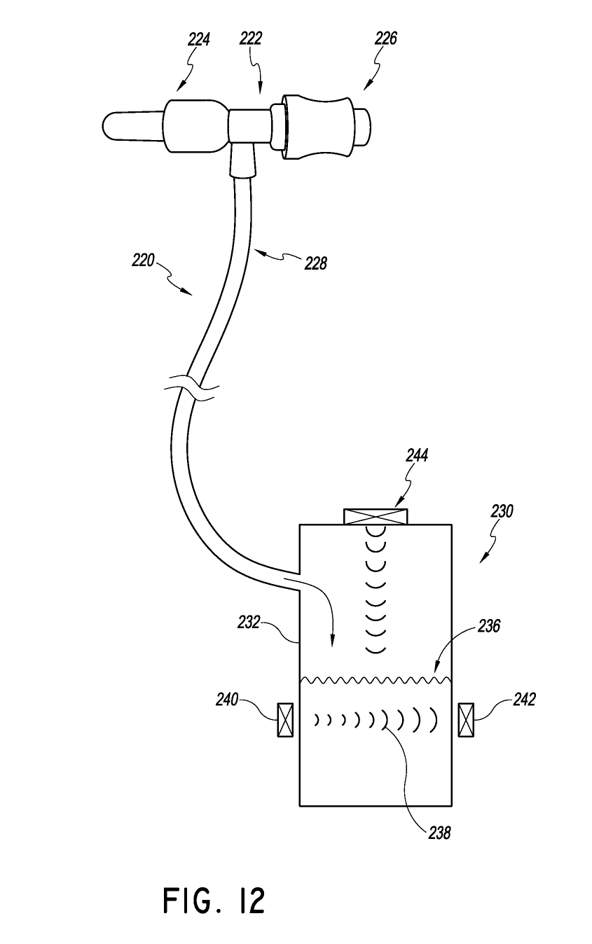

Referring to FIG. 12, there is schematically illustrated a side branch sampling system 220. The sampling system 220 comprises a manifold 222 which includes an effluent connector 224 such as a luer connector adapted to connect to the infusion port on the proximal end of a procedure catheter. The manifold 222 is additionally provided with an influent connector 226 such as an influent luer connector, for connection to the common flow line of fluids from all sources that will be introduced into the patient via the catheter. Manifold 222 additionally comprises a side branch 228, which directs a low volume flow utilized for sampling. The side branch 228 may be provided with an aperture or capillary tube of known dimensions, such that the volume of fluid exiting the infusion stream via the side branch 228 will be a known percentage of the total fluid flowing via the effluent connector 224 into the patient.

The manifold 222 and associated connectors and side branch line 228 can be dimensioned in a manner that provides minimal disruption to the operating site, and the side branch line 228 can be directed to a reservoir which is suspended from an IV pole, placed on the floor, or otherwise located out of the way of clinical personnel.

In the illustrated embodiment, side branch 228 empties into a fluid reservoir 230 having an internal capacity which greatly exceeds the likely total volume of fluid diverted via side branch line 228 during a typical procedure. The fluid reservoir 230 comprises a wall 232 defining an interior volume 234 for receiving liquid via side branch 228. Liquid may be characterized by a liquid level 236 which will rise slowly during the procedure.

The disposable fluid reservoir 230 may be provided with an optical path 238 which extends across at least a portion of the reservoir 230 and may be positioned below the liquid level 236. Optical path 238 extends between a light source 240 such as an LED, and a detector 242 such as a CCD or CMOS sensor, to measure light received from the light source. The length of the optical path 238 is known, such that absorption and concentration of the analyte can be determined in accordance with known techniques.

The reservoir 230 is additionally provided with a liquid level detector 244. In one implementation of the invention, liquid level detector 244 comprises an ultrasound source and receiver, for propagating an ultrasound signal and receiving reflections off of the surface of the liquid at liquid level 236. This enables determination of the liquid level in accordance with known calculations, and since the dimensions of the interior volume are known, permits calculation of total fluid volume diverted via side branch 228. From this, the total delivered dose of the analyte of interest (e.g. contrast media) to the patient via effluent connector 224 may be determined.

The fluid reservoir 230 may be configured in a disposable manner, such that it can be utilized a single time and then discarded along with the manifold 222 and associating tubing. In this implementation, the disposable fluid reservoir 230 would be configured for removable positioning within a housing, which carries the light source 240, sensor 242 and detector 244. Alternatively, the light source 240 sensor 242 and detector 244 may be secured to the wall 232 of the disposable fluid reservoir 230, and the entire assembly is configured for disposal following a single use. As with previous embodiments, the cumulative dose and other determined information can be wired or wireless ported to a display that may be mounted on a bedside stand or table, a wall, or to a portable device such as an iPad, laptop or cellular phone.

Due to variations in pressure, backflow of blood via effluent 224 and into the reservoir 230 may occur. If backflow blood absorbs sufficiently at 244 nm to mask absorption of the iodine based contrast media it may be desirable to measure transmission at a second wavelength which can be utilized to differentiate contrast from blood. As a further enhancement, it may be desirable to quantitatively add a dye to the contrast media which exhibits strong absorption in the UV visible range at a peak which is unaffected by the presence of blood.

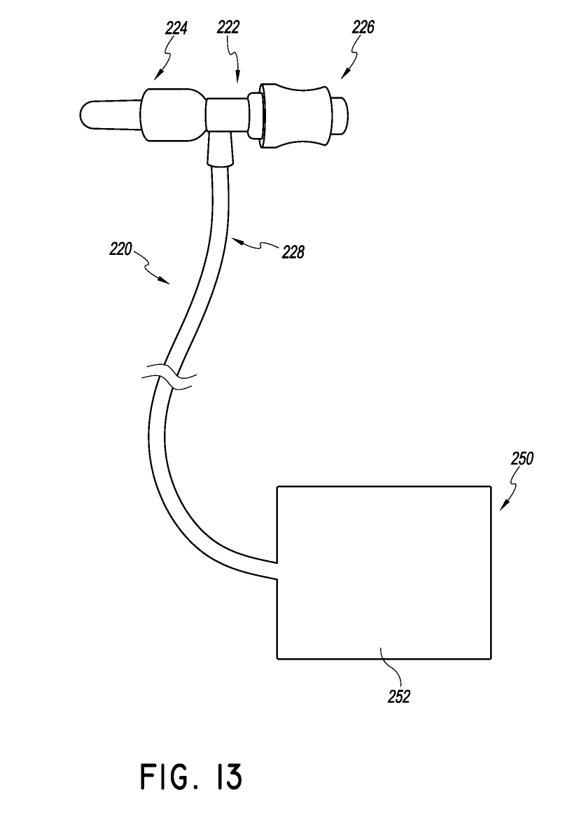

In accordance with a further implementation of the invention, a removable module 250 is placed in fluid communication with the common flow path 20. See, FIG. 13. The removable module may be placed in communication with the common flow path by a side branch 228, or the removable module may be placed directly in the common flow path 20 such that fluid enters an input on the removable module and exits an output to continue along the flow path 20.

The removable module 250 contains an extraction system 252 for extracting a measurable analyte of interest in the flow path 20. This may include specific binding partners, beads or other particulate material, open cell foam, or other structures or chambers for entrapping either whole fluid extending through flow path 20, or analytes of interest travelling through flow path 20.

The removable module 250 may be removed from flow path 20, and introduced into a reader which may be in the same room or in a different room. The reader may use any of a variety of technologies described elsewhere herein, to determine the concentration of contrast media contained in the removable module. Calculations can be conducted to thereafter determine the delivered dose of contrast media to the patient. For example, the removable module 250 may be exposed to a source of x-rays, to enable measurement of contrast media directly. Alternatively, contents of the removable module 250 may be eluted from the module such as by rinsing or other technique, and thereafter evaluated in a spectrophotometer or other analytical equipment.

This implementation of the invention therefore permits periodic determination of the delivered dose. The removable module 250 may be replaced in the common flow path 20, with or without a rinse or elution procedure. Alternatively, the removable module 250 may be configured as a one-time use device, such that a series of two or three or four or more removable modules may be successively utilized throughout the interventional procedure.

Specific features of the invention are shown in one or more of the drawings for convenience only, as each feature may be combined with other features in accordance with the invention. Alternative embodiments will be recognized by those skilled in the art and are intended to be included within the scope of the claims. Accordingly, the above description should be construed as illustrating and not limiting the scope of the invention. All such obvious changes and modifications are within the patented scope of the appended claims.

What is claimed is:

1. A system for measuring total delivered dose of contrast media during an intravascular procedure, comprising:
an influent connector, configured to connect to at least one source of fluid;
an effluent connector, configured to connect to an infusion port on a catheter;
a flow rate detector, configured to measure the rate of fluid flow through the system; and
an analyte detector, configured to detect the concentration of an analyte of interest flowing through the system.

2. A system for measuring total delivered dose as in claim 1, wherein the flow rate detector comprises a detector for determining change in refractive index.

3. A system for measuring total delivered dose as in claim 1, wherein the flow rate detector comprises a detector for determining thermal marker transit time.

4. A system for measuring total delivered dose as in claim 1, wherein the flow rate detector comprises a detector for determining change in electrical conductivity.

5. A system for measuring total delivered dose as in claim 1, wherein the analyte detector comprises a light source and a sensor.

6. A system for measuring total delivered dose as in claim 5, wherein the light source comprises a source of UV light.

7. A system for measuring total delivered dose as in claim 6, wherein the analyte detector is configured to determine absorption at about 244 nm.

8. A system for measuring total delivered dose as in claim 5, comprising an optical flow cell in fluid communication with the effluent connector, having the light source on a first side of the optical cell and the detector on an opposing side of the optical cell.

9. A system for measuring total delivered dose as in claim 8, wherein at least one of the light source and the sensor is attached to the optical cell.

10. A system for measuring total delivered dose as in claim 8, wherein at least one of the light source and the sensor is attached to a console, and the optical cell is configured for removable engagement with the console.

11. A system for measuring total delivered dose as in claim 10, wherein each of the light source and the sensor is attached to a console, spaced apart to define a cavity to receive the optical cell.

12. A system for measuring total delivered dose as in claim 1, wherein the source of fluid comprises a saline bag and at least one contrast injection port.

13. A system for measuring total delivered dose as in claim 1, wherein the light source comprises an LED.

14. A system for measuring total delivered dose as in claim 1, wherein the sensor comprises a CCD sensor.

15. A system for measuring total delivered dose as in claim 1, wherein the sensor comprises a CMOS sensor.

16. A system for measuring total delivered dose as in claim 1, further comprising a display, for displaying an indicium of cumulative delivered dose of the analyte of interest.

17. A system for measuring total delivered dose as in claim 1, further comprising a memory configured to store total delivered dose data from a plurality of discrete intravascular procedures.

18. A method of monitoring total delivered dose of contrast media injection during an intravascular procedure, comprising:
providing a system for measuring total delivered dose of contrast media, the system comprising a flow rate detector, an analyte detector, an effluent port connected to an infusion port on a catheter, a fluid source and an injection port and tubing configured to merge fluid from the fluid source and the injection port into a common flow path which flows through the flow rate detector and analyte detector; and
infusing fluid from the fluid source and at least one bolus of contrast media through the injection port;
wherein the system determines the total, cumulative amount of contrast media delivered through the common flow path during the intravascular procedure.

19. A method of monitoring total delivered dose of contrast media injection as in claim 18, wherein the procedure comprises an angioplasty procedure.

20. A method of monitoring total delivered dose of contrast media injection as in claim 18, wherein the procedure comprises an angioplasty, stent placement, thrombolytic procedure, embolization procedure, electrophysiology procedure or other endovascular procedure in the heart, brain or peripheral arteries.

21. A method of monitoring total delivered dose of contrast media injection as in claim 18, wherein the procedure comprises a heart valve replacement procedure.

22. A method of monitoring total delivered dose of contrast media injection as in claim 18, wherein the procedure comprises a heart valve repair procedure.

23. A method of monitoring total delivered dose of contrast media injection as in claim 18, wherein the procedure comprises an abdominal aortic artery graft deployment procedure.

24. A dosimeter for determining cumulative delivered dose of an analyte of interest, comprising:
a manifold, having an influent connector for connection to a common flow path of liquid from at least one source;
an effluent connector in fluid communication with the manifold, for connection to an infusion port on a catheter;
a side branch configured to divert a portion of fluid flowing through the manifold;
a container in fluid communication with the side branch, for receiving the diverted portion of fluid.

25. A dosimeter as in claim 24, further comprising at least one analyte sensor for sensing an indicium of concentration of an analyte of interest.

26. A dosimeter as in claim 24, further comprising a light source spaced apart from a sensor along an optical path through the container.

27. A dosimeter as in claim 26, further comprising a sensor for determining total volume of liquid in the container.

28. A dosimeter as in claim 27, wherein the volume sensor comprises an ultrasound transducer.

29. A dosimeter as in claim 24, wherein the light source and the sensor are carried by the container.

30. A dosimeter as in claim 24, wherein the light source and the sensor are carried by a console configured to removably receive the container.
